(19) United States
(12) Patent Application Publication
(10) Pub. No.: US 2025/0099189 A1
(43) Pub. Date: Mar. 27, 2025

(54) SYSTEMS AND METHODS FOR PARAMETERIZING MEDICAL PROCEDURES

(71) Applicant: INTUITIVE SURGICAL OPERATIONS, INC., Sunnyvale, CA (US)

(72) Inventors: Anthony M. Jarc, Johns Creek, GA (US); Joey Chau, Cupertino, CA (US); May Quo-Mei Liu, Rougemont, NC (US)

(21) Appl. No.: 18/847,143

(22) PCT Filed: Mar. 14, 2023
(86) PCT No.: PCT/US2023/064335

§ 371 Date: Sep. 13, 2024

Related U.S. Application Data
(60) Provisional application No. 63/320,538, filed on Mar. 16, 2022.

(51) Int. Cl. A61B 34/30; G16H 10/60; G16H 20/40

(52) U.S. Cl. CPC A61B 34/30; G16H 10/60; G16H 20/40; A61B 2034/305

(57) ABSTRACT

A robot-assisted medical system comprises a manipulator arm, an operator console, a medical instrument coupled to the manipulator arm, and a control system in communication with the operator console and the manipulator arm. The control system comprises a processor and a memory comprising machine readable instructions. When executed by the processor, the machine readable instructions may cause the control system to receive a plurality of medical procedure records, identify a set of characteristic actions common among the plurality of medical procedure records, and determine at least one parameter for each characteristic action in the set of characteristic actions.

### CROSS-REFERENCED APPLICATIONS

This application claims priority to and benefit of U.S. Provisional Application No. 63/320,538, filed Mar. 16, 2022 and entitled “Systems and Methods for Parameterizing Medical Procedures,” which is incorporated by reference herein in its entirety.

### FIELD

The present disclosure is directed to systems and methods for parameterizing medical procedures.

### BACKGROUND

Minimally invasive medical techniques are intended to reduce the amount of tissue that is damaged during diagnostic or surgical procedures, thereby reducing patient recovery time, discomfort, and harmful side effects. Such minimally invasive techniques may be performed through one or more surgical incisions or through natural orifices in a patient anatomy. Through these incisions or natural orifices, clinicians may insert minimally invasive medical instruments to conduct medical procedures by manually or by a robot-assisting actuation of the instrument. Systems and methods are needed for determining parameters associated with characteristic actions of the medical procedures.

### SUMMARY

Examples of the invention are summarized by the claims that follow the description. Consistent with some examples, a method may comprise receiving a plurality of medical procedure records. Each of the plurality of medical procedure records may be generated during a medical procedure performed with a robot assisted medical system. The method also includes identifying a set of characteristic actions common among the plurality of medical procedure records and determining at least one parameter for each characteristic action in the set of characteristic actions.

In some examples, a robot-assisted medical system may comprise a manipulator arm, an operator console, a medical instrument coupled to the manipulator arm, and a control system in communication with the operator console and the manipulator arm. The control system may comprise a processor and a memory comprising machine readable instructions that, when executed by the processor, cause the control system to receive a plurality of medical procedure records and identify a set of characteristic actions common among the plurality of medical procedure records. The instructions may also cause the control system to determine at least one parameter for each characteristic action in the set of characteristic actions.

In some examples, a method may comprise receiving a medical procedure record, identifying one or more actions in the medical procedure record and determining at least one parameter for each of the one or more actions.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a schematic illustration of a medical procedure record, according to some embodiments.

FIG. 2 is a flowchart illustrating measurement and record systems for parameterizing a medical procedure.

FIG. 3 is a flowchart illustrating a method for parameterizing a medical procedure, according to some embodiments.

FIG. 4 is a schematic illustration of a parameterized medical procedure record, according to some embodiments.

FIG. 5 is a flowchart illustrating a method for parameterizing a medical procedure, according to some embodiments.

FIG. 6 is a schematic view of a robot-assisted medical system according to some embodiments.

FIG. 7 is a perspective view of a manipulator assembly according to some embodiments.

FIG. 8 is a perspective view of an operator input system according to some embodiments.

Embodiments of the present disclosure and their advantages are best understood by referring to the detailed description that follows. It should be appreciated that like reference numerals are used to identify like elements illustrated in one or more of the figures for purposes of illustrating but not limiting embodiments of the present disclosure.

### DETAILED DESCRIPTION

Systems and methods are provided for parameterizing medical procedures. From aggregated medical procedure records, characteristic actions from the medical procedural records may be identified. Parameters associated with the characteristic actions may also be determined from the procedural records. The parameters may be measured or recorded aspects of the procedure, including pre-interventional, interventional, and post-interventional stages of the procedure. The measured and recorded parameters for multiple procedures of a same type may be aggregated and associated with procedure outcomes.

During a medical procedure, sensor systems, measurement systems, and/or recording systems may capture and store data records in a medical procedure record. In some examples, the medical procedure record may capture information about a medical procedure performed with a robot-assisted medical system, with a laparoscopic medical system, with a manual medical device, or with a combination of systems and devices. Each medical procedure record may capture information about a medical procedure performed on a patient by a single clinician or a team of medical professionals. Medical procedure records may be generated, for example, from procedures performed on a patient, during a training session with synthetic tissue structures, or during a prior computer-generated simulation.

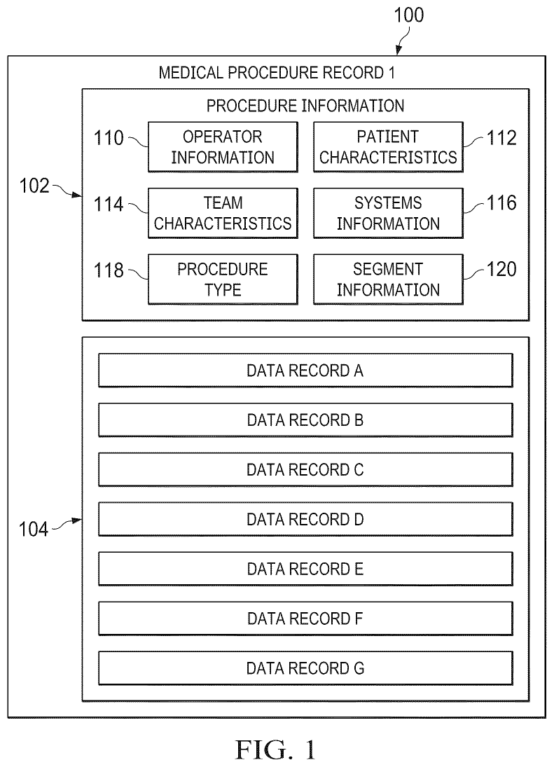

In some examples, as shown in the schematic illustration of FIG. 1, a medical procedure record 100 includes procedure information 102 and data records 104 from the medical procedure. The procedure information 102 may include operator information 110 including the clinician identification information, training history, experience, preferences, and/or other information related to one or more clinicians involved in the medical procedure. The operator may be, for example, a surgeon, a surgical trainee, an expert or guide medical practitioner. The procedure information 102 may include patient characteristics 112 including patient age, gender, height, weight, medical history and/or other information related to the unique patient on which the medical procedure was performed. The patient characteristics may be specific to a procedure such as a level of tissue inflammation, abnormalities in tissue thickness or toughness, size of hernia. The patient characteristics may also include anomalies such as cysts, additional lymph nodes, and tumor size, location, or complexity. The procedure information may include team characteristics 114 for the team members and support staff involved in the medical procedure including identification information, role in the procedure, training history, experience, preferences, or other information related to the personnel performing the procedure. The procedure information 102 may also include system information 116 about the system or systems used to perform the procedure. The systems may include sensors or recording systems that capture information such as settings or sensed parameters for the system during a procedure. Systems may include a robot-assisted medical system or components thereof, a laparoscopic medical system, a manual medical device, and/or support or peripheral systems. The system information 116 may include manufacturer, model, serial number, time in service, time since last maintenance, count of use cycles, maintenance history, calibration information, or other information related to systems used to perform the medical procedure. The procedure information 102 may also include procedure type 118 which includes information about the type of medical procedure. Medical procedure type may include abdominal, cardiac, colorectal, gynecological, neurological, head/neck, pulmonological, thoracic, urologic, or other category or subcategory of anatomic system involved in the medical procedure. The procedure information 102 may also include segment information 120. Segment information 120 may include information about subdivided portions of the medical procedure. For example, procedure segments may include sequences or groups of actions associated with ablation, stapling, suturing, dissection, tissue resection, anastomosis, camera control, instrument wrist control, setting changes, or tool changes that occur once or multiple times during a medical procedure. In some examples, procedure information may also include other information about the procedure including, for example, the date on which the procedure occurred, the time and duration of the procedure, and/or the location and facility identification where the procedure occurred.

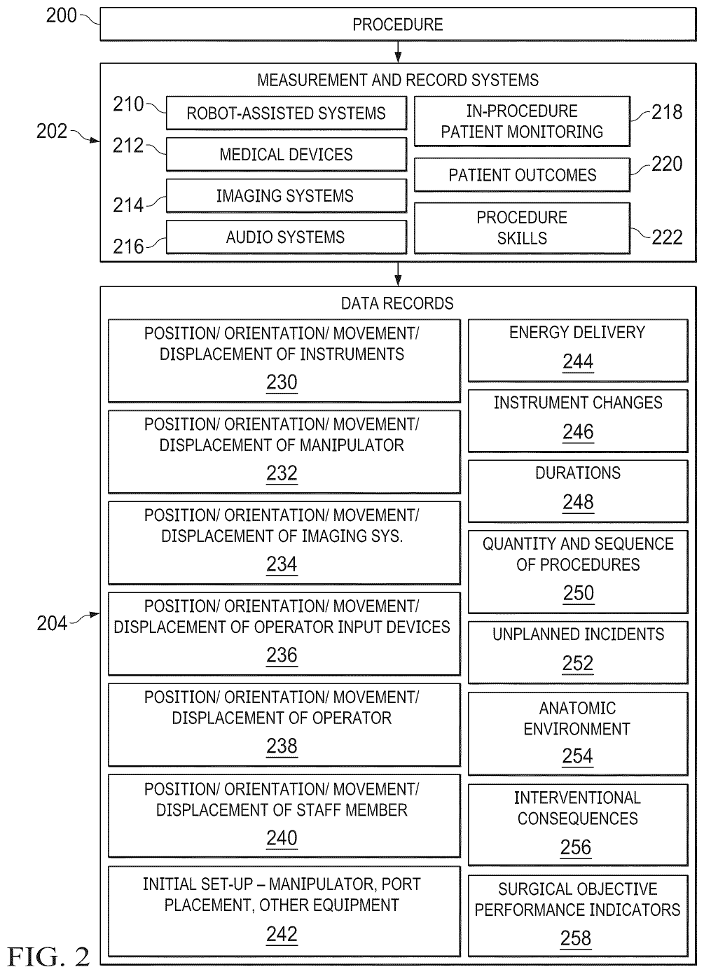

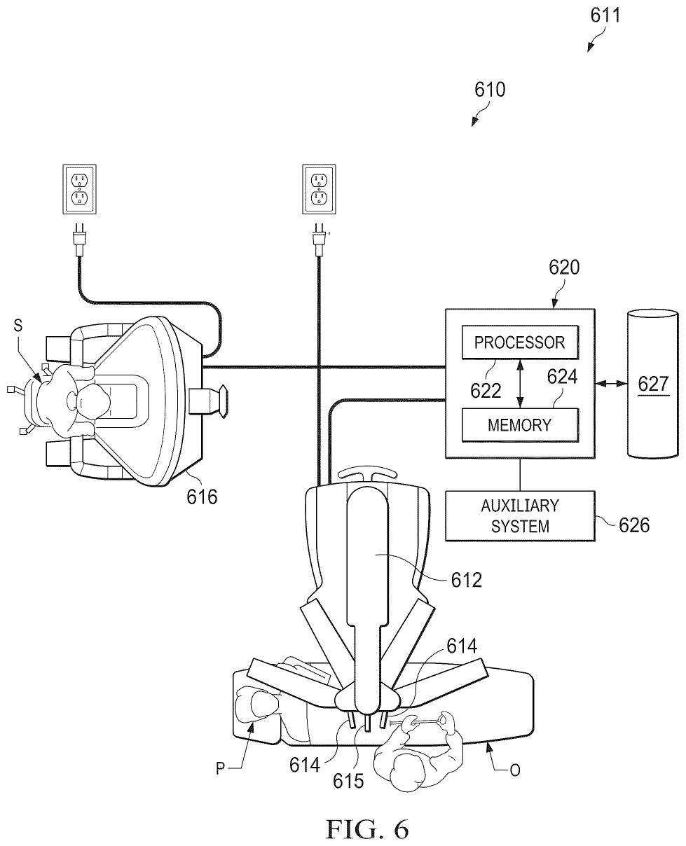

The medical procedure record 100 also includes data records 104 captured during the medical procedure. In this example, data records 104 may include data records A-G. In some examples, as illustrated in FIG. 2 a medical procedure 200 may be documented using a variety of measurement and record systems 202 to generate data records 204 (e.g., the data records 104) associated with the procedure 200. Measurement and record systems 202 may include, for example one or more sensor systems 210 associated with a robot-assisted medical system (e.g., medical system 610 of FIG. 6). In some examples, the sensor system 210 may include position, orientation, motion, and/or displacement sensor systems for manipulators or instruments coupled to manipulators of the robot-assisted medical system. In some examples, the sensor system 210 may include force sensor systems, clocks, motor encoders, energy usage sensors, user eye-tracking sensors, or other sensor systems that measure and/or record data about the manipulator or instruments. Measurement and record systems 202 may also or alternatively include one or more sensor systems 212 associated with monitored medical devices used in the medical procedure 200. In some examples, the sensor systems 212 may track position, orientation, motion, displacement, force, energy usage, duration of use, and/or other measures associated with the medical devices.

Measurement and record systems 202 may also or alternatively include one or more imaging systems 214 used during the medical procedure 200. In some examples, the imaging systems 214 may be in vivo imaging systems such as endoscopic imaging systems or ultrasound imaging systems used during the procedure 200. In some examples, the imaging systems 214 may be ex vivo imaging systems for the patient anatomy such as computed tomography (CT) imaging systems, magnetic resonance imaging (MRI) imaging systems, or functional near-infrared spectroscopy (fNIRS) imaging systems used during the procedure 200. In some examples, the imaging systems 214 may be environment imaging systems such as optical imaging systems that track the position and movement of manipulators, instruments, equipment, and/or personnel in the environment of the patient during the procedure 200.

Measurement and record systems 202 may also or alternatively include one or more audio systems 216 used during the medical procedure 200. The audio systems may capture and record audio from the personnel in the medical area of the procedure 200, the operator performing the procedure 200, the patient, and/or equipment in the medical area of the procedure. Measurement and record systems 202 may also or alternatively include one or more in-procedure patient monitoring systems 218 used during the medical procedure 200. The patient monitoring systems 218 may include, for example, respiration, cardiac, blood pressure, anesthesia, insufflation, and/or patient/table orientation monitoring systems. Measurement and record systems 202 may also or alternatively include one or more patient outcome record systems 220 that may be referenced after the procedure 200 is complete. Patient outcome record systems 220 may record information about post-procedure hospitalization duration, complications, positive outcomes, negative outcomes, mortality, or other post-procedure information about the patient. Measurement and record systems 202 may also or alternatively include one or more procedure skills record systems 222 that capture and record objective performance indicators for the clinician that performs the procedure 200.

The data records 204 may include the data generated by the measurement and record systems 202. For example, data records 230 may record the position, orientation, movement, and/or displacement of instruments (e.g., instruments 614) controlled by a robot assisted manipulator or by manual operation. In some examples, data records 232 may record the position, orientation, movement, and/or displacement of a robot-assisted manipulator assembly (e.g., 612) including any arms of the manipulator during the procedure 200. In some examples, data records 234 may record the position, orientation, movement, and/or displacement of an imaging system, such as an endoscopic or other in vivo or ex vivo imaging system, during the procedure 200. In some examples, data records 236 may record the position, orientation, movement, and/or displacement of an operator input device (e.g., 636) during the procedure 200. In some examples, data records 238 may record the position, orientation, movement, and/or displacement of an operator (e.g., surgeon S) directing the control of an instrument during the procedure 200. For example, the data records 238 may record motion of the operator's hands or track head disengagement from an operator console. In some examples, data records 240 may record the position, orientation, movement, and/or displacement of one or more members of a medical team involved with the procedure 200. In some examples, data records 242 may record aspects of the initial set-up of the procedure 200, including the position and arrangement of the robot-assisted manipulator, patient port placement, and the location of peripheral equipment. In some examples, data records 244 may include records of the location, frequency, and amount of energy provided to or delivered by instruments (e.g. ablation instruments) during the procedure 200. In some examples, data records 246 may include records of instrument changes during the procedure 200. In some examples, data records 248 may include time-based records that capture dwell times, idle times, and/or duration or speed of an action during the procedure 200. In some examples, data records 250 may capture aspects of the workflow including the quantity and/or sequence of actions during the procedure 200. For example, the data records 250 may include sequences of position, orientation, movements, and/or displacements associated with a discrete activity. In some examples, data records 252 may capture errors, difficulties, incidents, or other unplanned episodes, such as manipulator arm collisions, during the procedure 200 conditions leading to conversions during the procedure from a robot-assisted surgery to an open surgery, conditions leading to conversions during the procedure from a robot-assisted surgery to a laparoscopic surgery, or conditions leading to conversions during the procedure from a laparoscopic surgery to an open surgery. In some examples, data records 254 may capture aspects of the anatomic environment including size of organs, incisions, and/or treatment delivery areas. Other aspects of the anatomic environment that may be recorded include pelvic width, distance between anatomic structures, and/or locations of vasculature. In some examples, data records 256 may include interventional consequences such as measures of bleeding, smoke, tissue movement, and or tissue color change. In some examples, data records 258 may include a catalog of the key skills to perform the procedure 200, the relevant object performance indicators for experienced clinicians that perform the same type of procedure, and objective performance indicators of the clinician who performed the procedure 200.

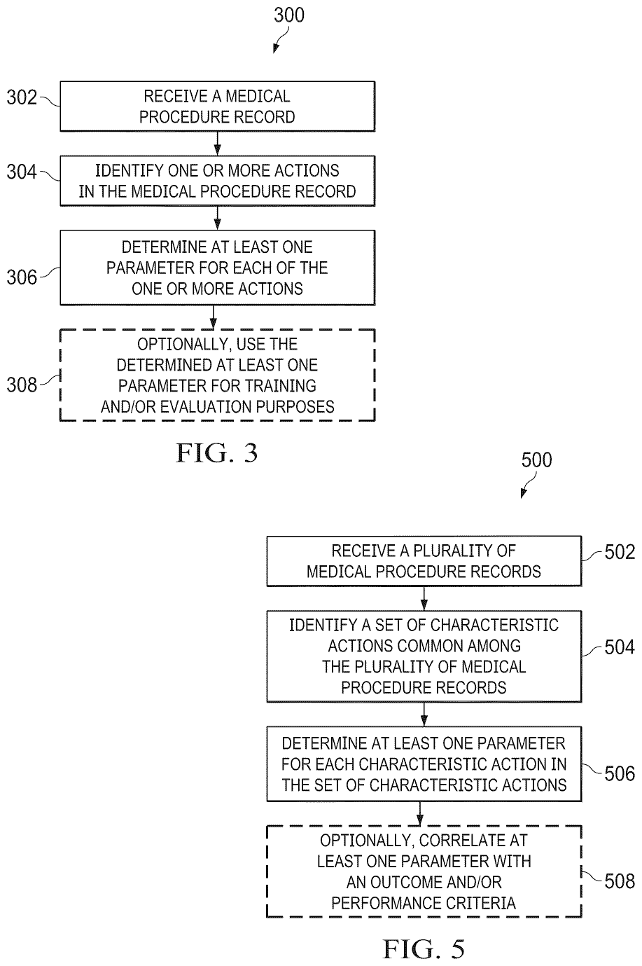

FIG. 3 illustrates a method 300 for parameterizing a medical procedure. The method 300 is illustrated as a set of operations or processes. The processes illustrated in FIG. 3 may be performed in a different order than the order shown in FIG. 3, and one or more of the illustrated processes might not be performed in some embodiments of method 300. Additionally, one or more processes that are not expressly illustrated in FIG. 3 may be included before, after, in between, or as part of the illustrated processes. In some embodiments, one or more of the processes of method 300 may be implemented, at least in part, in the form of executable code stored on non-transitory, tangible, machine-readable media that when run by one or more processors (e.g., the processors of a control system) may cause the one or more processors to perform one or more of the processes.

At a process 302, a medical procedure record may be received. For example, the medical procedure record may be received at a processor (e.g., processor 620 of medical system 610). In some examples, the medical procedure record may be received from a memory device (e.g., a memory 624 of medical system 610). The received medical record may be, for example, the medical record 100.

At a process 304, one or more actions in the medical procedure may be identified through analysis of the medical procedure record (e.g., procedure information 102 and/or data records 104). In some examples, the identification of the actions may be determined by computer analysis. Additionally or alternatively, a human analyst may identify one or more actions. In some examples, actions of a procedure may include a single action such as grasping tissue with a gripping instrument. In some examples, an action of a procedure may include a sequence of movements or behaviors. For example, the action of creating a stitch may involve the movements of gripping a suturing filament with an instrument, rotating a wrist joint of the instrument which grips the suturing filament, and releasing the suturing filament.

At a process 306, at least one parameter may be determined for each action of the medical procedure. The parameter may be determined, for example, from one or more of the data records (e.g., data records 104, 204) or procedural information records (e.g., procedural information 102) from the medical procedure record. Multiple parameters may be determined for each action, and thus a medical procedure with multiple actions may include many parameters. In some examples, parameters associated with the action of grasping tissue may include the identity of the instrument used for grasping the tissue, the identity of the manipulator arm used to control the instrument, the force applied by the instrument, the duration of the application of the force, and/or a measure of interventional consequences such as tissue color change.

In some examples, parameters associated with the action of suturing tissue may be determined from the data records or procedural information records associated with a series of steps in performing the act of suturing. For example, parameters associated with suturing may include the identity of the instrument used for grasping the suturing filament, the identity of the manipulator arm used to control the instrument, the force applied to grasp the filament, the rotation of the wrist joint of the instrument, the duration of time to complete the rotation, the position and orientation of the instrument at release of the filament, and/or a measure of interventional consequences such as bleeding.

In some examples, parameters associated with the action of delivering energy to tissue may be determined from the data records or procedural information records associated with the action of delivering energy. For example, parameters associated with delivering energy to tissue may include the identity of the instrument used for ablating tissue, the identity of the manipulator arm used to control the ablation instrument, the measure of energy delivered to the tissue, the movement of the instrument, the duration of time to complete the ablation, the number of locations at which the energy was delivered, and/or a measure of interventional consequences such as smoke generated during the action.

In some examples, parameters associated with the action of camera control may be determined from the data records or procedural information records associated with the action of camera control or with camera position and orientation. For example, parameters associated with camera control may include a motion of other instruments immediately preceding or following the movement of an endoscope (e.g., the instrument motion may indicate why the change in camera was needed), the change in position and/or orientation of a distal end of the endoscope, and/or a change in focus of the endoscope.

In some examples, parameters associated with the action of an instrument motion may be determined from the data records or procedural information records associated with the instrument motion. For example, parameters associated with moving the instrument may include the measures of position, orientation, speed, and/or displacement of an end effector of the instrument. Parameters may also include position, orientation, speed, and/or displacement of a manipulator arm to which the instrument is coupled. Parameters may also include error indicators or other measures of arm collision.

In some examples, parameters associated with the action of an operator may be determined from the data records or procedural information records associated with the operator console. For example, parameters associated with operator engagement may include data records associated with eye gaze, head engagement with the operator console, duration of time to perform an action, the hand used to control an instrument motion, and or the motion of an operator input device.

In some examples, the determined parameters may be clinically relevant parameters such as duration or motion data that provide information about speed or efficiency of motion and that may provide an indication of operator skill. In some examples, the determined parameters may be demarcation parameters that provide an indication of a stage or point in a procedure. For example, a data record of a foot pedal input may be a demarcation parameter that indicates the beginning of a camera control action in which the angle or position of an endoscope is adjusted. A subsequent second data record of a food pedal input may be a demarcation parameter that indicates the end of the camera control action and a return to an instrument following mode.

At an optional process 308, the determined parameter(s) may be used for a variety of purposes including training, evaluation, procedure improvement assessment, or other development or analytic purposes. For example, the determined parameters may be used to conduct a training procedure to strengthen the operator's skill competencies. In some examples, one or more parameterized procedures may be used to create simulation exercises that address the development needs of the clinician. A simulation exercise may be customized, scaled, or otherwise adapted to the operator's schedule of upcoming procedures. In some examples, the experience may be customized to include remediation exercises to correct performance or to include increasingly difficult exercises to expand the user's capabilities. A simulation exercise may allow the user to virtually experience one or more aspects of a single parameterized procedure or of a hybrid of more than one parameterized procedures. The simulation exercise may include a simulated user interface that provides visual, audio, and/or haptic simulation of the procedure. The simulation exercise may include some or all the parameters from the parameterized procedure, based on the data records obtained during the procedure. The simulation exercise may simulate the robot-assisted medical systems and instruments, laparoscopic instruments, and/or open procedure instruments used in the parameterized procedure. Image data, audio data, force transmission data, patient monitoring data, and/or patient outcome data recorded or gathered and parameterized from the procedure may be included in the simulation exercise. In some examples, image data, audio data, force transmission data, patient monitoring data, and/or patient outcome data may be artificially generated and included in the simulation exercise to create a synthetic or hybrid synthetic-recorded environment and experience. The simulation exercise may be interactive and responsive to user inputs. In some examples, the simulation exercise may be presented to the user at a simulated user console that includes user interface components of an operator input system (e.g., operator input system 616) including display systems, audio systems, and user input control devices. In some examples, the simulation exercise may be presented to the user at an actual user console (e.g. operator input system 616) that is operating in a simulation mode. In some examples, the simulation exercise may be adapted for presentation to the user on a laptop, tablet, phone or other user input device that may include a display, user input control devices, a control system, a memory, and/or other components that support the visual, audio, and/or haptic user experience. In some examples, a simulation may dynamically adapted based on user preference. In some examples, the simulation may include an inanimate anatomic model or a synthetic tissue model customized and built for the simulation. For example, a synthetic model may include a custom anatomical defect or customized instrument port placements relative to a defect.

In some examples, the determined parameters may be used to evaluate the operator. Such evaluations may include comparisons to comparable parameters from different users, experts, or standards for the same or similar actions or procedures. In some examples the determined parameters may be used to identify inefficiencies, delays, equipment issues, environment issues, surgical team issues or other issues that may be addressed to improve future procedures.

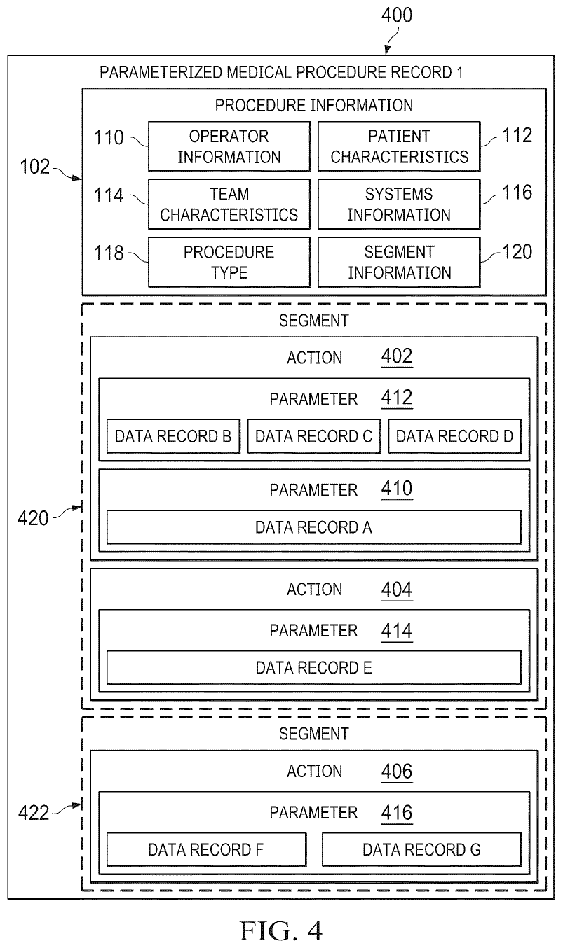

FIG. 4 is a schematic illustration of a parameterized medical procedure record 400 based on the initial medical procedure record 100. The parameterized medical procedure record 400 may include procedure information 102 and the data records A-G from the medical procedure. In parameterized medical procedure record 400, the data records A-G have been associated with actions 402, 404, 406 and parameters 410, 412, 414, 416 through the parameterization method 300. In this example the actions and parameters have, optionally, been further grouped into segments 420, 422. For example, procedure segments may include sequences or groups of actions associated with ablation, stapling, suturing, dissection, tissue resection, anastomosis, camera control, instrument wrist control, setting changes, or tool changes that occur once or multiple times during a medical procedure. In this example, a procedure may include segment 420 and segment 422. The segment 420 may include action 402 and 404. The action 402 may include two parameters 410 and parameter 412. The parameter 410 is or is determined from the single data record A. The parameter 412 is or is determined from three data records B-D. The action 404 may include a single parameter 414 that is or is determined from the data record E. The segment 422 includes a single action 406, and the action 406 includes a single parameter 416. The parameter 413 is or is determined from data record F and data record G.

In some examples the procedure record 400 may include segment 420 that is a tissue resection segment and segment 422 that is an ablation segment. The suturing segment 420 may include the actions of cutting tissue at action 402 and moving the cut tissue at action 404. The cutting action 402 may include a parameter 410 that includes a data record A that includes identification information for the cutting instrument. The cutting action 402 may include a parameter 412 that includes a data record B that includes the position and orientation of the end effector of the cutting instrument at the start of the cutting, a data record C that includes the position and orientation of the end effector of the cutting instrument at the conclusion of the cutting, and a data record D that includes a time duration between the start and conclusion of the cutting. The tissue moving action 404 may include the parameter 414 that includes a data record E that includes a distance the tissue is moved. The ablation segment 422 incudes a single action 406 of ablating tissue which includes the parameter 416 that is associated with a data record F for a power level and a data record G for a duration.

FIG. 5 illustrates a method 500 for parameterizing a type of medical procedure from a plurality of medical procedures. The method 500 is illustrated as a set of operations or processes. The processes illustrated in FIG. 5 may be performed in a different order than the order shown in FIG. 5, and one or more of the illustrated processes might not be performed in some embodiments of method 500. Additionally, one or more processes that are not expressly illustrated in FIG. 5 may be included before, after, in between, or as part of the illustrated processes. In some embodiments, one or more of the processes of method 500 may be implemented, at least in part, in the form of executable code stored on non-transitory, tangible, machine-readable media that when run by one or more processors (e.g., the processors of a control system) may cause the one or more processors to perform one or more of the processes.

At a process 502, a plurality of medical procedure records may be received. For example, the medical procedure records may be received at a processor (e.g., processor 620 of medical system 610). In some examples, the medical procedure records may be received from a memory device (e.g., a memory 624 of medical system 610). In some examples, as few as one medical record (e.g., medical record 100) may be received, but in other examples dozens, hundreds, thousands, or millions of medical procedure records that include procedural information and data records may be received.

At a process 504, a set of characteristic actions common among the plurality of medical procedure records may be identified through analysis of the procedure information (e.g., procedure information 102), data records (e.g., data records 104), segment information (e.g., segment information 120, 420, 422), action information from the plurality of medical procedure records (e.g., actions 402, 404, 406), or parameter information from the plurality of medical procedure records (e.g. parameters 410, 412, 414, 416). The characteristic action may include the action of an instrument or category of instruments, an instrument manipulator or category of instrument manipulators, an operator console component or category of operator console components, or other system, device, or component common among the plurality of medical procedure records. For example, a retractor instrument may be a category or type of instrument, although not the exact instrument, that is utilized within one or more of the plurality of medical procedure records. Within the plurality of medical records, characteristic actions may include a single characteristic action such as grasping tissue with a gripping instrument. Additionally or alternatively, a characteristic action may include a sequence of movements or behaviors. For example, a characteristic action found to be common among the plurality of medical procedure records may be the action of creating a stitch which may involve the movements of gripping a suturing filament with an instrument, rotating a wrist joint of the instrument which grips the suturing filament, and releasing the suturing filament. In other examples, characteristic action may include, but are not limited to, clamping the jaws of an end effector, delivering energy through an ablation instrument, changing the pose of an endoscopic imaging instrument, or moving an operator input device at an operator console.

At a process 506, at least one parameter may be determined for each characteristic action in the set of characteristic actions. The parameter may be determined, for example, from one or more of the data records (e.g., data records 104, 204) or procedural information records (e.g., procedural information 202) from the plurality of medical procedure records.

In some examples, parameters associated with the characteristic action of grasping tissue may include the identity of the various instrument used for grasping the tissue in the plurality of medical procedure records, the identity of the manipulator arm used to control the instrument in the plurality of medical procedure records, the force applied by the instrument in the plurality of medical procedure records, the duration of the application of the force, and/or a measure of interventional consequences such as tissue color change in the plurality of medical procedure records.

In some examples, parameters associated with the characteristic action of suturing tissue may be determined from the data records or procedural information records associated with a series of steps in performing the act of suturing. For example, parameters associated with suturing may include the identities of the instrument used for grasping the suturing filament in the plurality of medical procedure records, the identities of the manipulator arm used to control the instrument in the plurality of medical procedure records, the forces applied to grasp the filament, the rotation of the wrist joint of the instrument in the plurality of medical procedure records, the durations of time to complete the rotation in the plurality of medical procedure records, the positions and orientations of the instrument at release of the filament in the plurality of medical procedure records, and/or measures of interventional consequences such as bleeding in the plurality of medical procedure records.

In some examples, parameters associated with the action of delivering energy to tissue may be determined from the data records or procedural information records associated with the characteristic action of delivering energy. For example, parameters associated with delivering energy to tissue may include the identity of the instrument used for ablating tissue in the plurality of medical procedure records, the identity of the manipulator arm used to control the ablation instrument in the plurality of medical procedure records, the measure of energy delivered to the tissue in the plurality of medical procedure records, the movement of the instrument, the duration of time to complete the ablation in the plurality of medical procedure records, the number of locations at which the energy was delivered in the plurality of medical procedure records, and/or a measure of interventional consequences such as smoke generated during the action in the plurality of medical procedure records.

In some examples, parameters associated with the characteristic action of camera control may be determined from the data records or procedural information records associated with the action of camera control. For example, parameters associated with camera control may include a motion of other instruments immediately preceding or following the movement of an endoscope in the plurality of medical procedure records, the change in position and/or orientation of a distal end of the endoscope in the plurality of medical procedure records, and/or a change in focus of the endoscope in the plurality of medical procedure records.

In some examples, parameters associated with the characteristic action of an instrument motion may be determined from the data records or procedural information records associated with the instrument motion. For example, parameters associated with moving the instrument may include the measures of position, orientation, speed, and/or displacement of an end effector of the instrument in the plurality of medical procedure records. Parameters may also include position, orientation, speed, and/or displacement of a manipulator arm to which the instrument is coupled in the plurality of medical procedure records. Parameters may also include error indicators or other measures of arm collision in the plurality of medical procedure records.

In some examples, parameters associated with the characteristic action of an operator may be determined from the data records or procedural information records associated with the operator console. For example, parameters associated with operator engagement may include data records associated with eye gaze, head engagement with the operator console, duration of time to perform an action, the hand used to control an instrument motion, and or the motion of an operator input device in the plurality of medical procedure records.

In some examples, the determined parameters may be clinically relevant parameters such as duration or motion data that provide information about speed or efficiency of motion and that may provide an indication of operator skill. In some examples, the determined parameters may be demarcation parameters that provide an indication of a stage or point in a procedure.

With the characteristic action identified and the medical procedures parameterized, the medical procedures, segments, or actions may, optionally, be evaluated for quality, efficiency, patient outcome or other evaluation criteria. At an optional process 508, for example, the determined parameter(s) may correlated with an outcome or performance criteria. For example, parameters associated with low patient morbidity, low procedure duration, minimized camera pose changes, minimized surgical attendant intervention, minimized manipulator arm collisions, or quality indicia may be identified through the plurality of medical procedure records. These parameters may be used, for example, for evaluation and training of operators and/or development of new or improved procedures. As described in International Application No. PCT/US2023/064324 [P06470-WO], which is incorporated by reference herein in its entirety, parameterized medical procedures may be referenced in a medical skill development system to generate customized medical simulations.

The medical procedures described herein may be performed with a variety of manual or robot-assisted technologies. FIGS. 6-8 together provide an overview of a robot-assisted medical system 610 that may be used in, for example, medical procedures including diagnostic, therapeutic, or surgical procedures. The medical system 610 is located in a medical environment 611. The medical environment 611 is depicted as an operating room in FIG. 6. In other embodiments, the medical environment 611 may be an emergency room, a medical training environment, a medical laboratory, or some other type of environment in which any number of medical procedures or medical training procedures may take place. In still other embodiments, the medical environment 611 may include an operating room and a control area located outside of the operating room.

In one or more embodiments, the medical system 610 may be a teleoperational medical system that is under the teleoperational control of a surgeon. In alternative embodiments, the medical system 610 may be under the partial control of a computer programmed to perform the medical procedure or sub-procedure. In still other alternative embodiments, the medical system 610 may be a fully automated medical system that is under the full control of a computer programmed to perform the medical procedure or sub-procedure with the medical system 610. One example of the medical system 610 that may be used to implement the systems and techniques described in this disclosure is the da Vinci® Surgical System manufactured by Intuitive Surgical Operations, Inc. of Sunnyvale, California.

As shown in FIG. 6, the medical system 610 generally includes an assembly 612, which may be mounted to or positioned near an operating table O on which a patient P is positioned. The assembly 612 may be referred to as a patient side cart, a surgical cart, or a surgical robot. In one or more embodiments, the assembly 612 may be a teleoperational assembly. The teleoperational assembly may be referred to as, for example, a manipulating system and/or a teleoperational arm cart. A medical instrument system 614 and an endoscopic imaging system 615 are operably coupled to the assembly 612. An operator input system 616 allows a surgeon S or other type of clinician to view images of or representing the surgical site and to control the operation of the medical instrument system 614 and/or the endoscopic imaging system 615.

The medical instrument system 614 may comprise one or more medical instruments. In embodiments in which the medical instrument system 614 comprises a plurality of medical instruments, the plurality of medical instruments may include multiple of the same medical instrument and/or multiple different medical instruments. Similarly, the endoscopic imaging system 615 may comprise one or more endoscopes. In the case of a plurality of endoscopes, the plurality of endoscopes may include multiple of the same endoscope and/or multiple different endoscopes.

The operator input system 616 may be located at a surgeon's control console, which may be located in the same room as operating table O. In one or more embodiments, the operator input system 616 may be referred to as a user control system. In some embodiments, the surgeon S and the operator input system 616 may be located in a different room or a completely different building from the patient P. The operator input system 616 generally includes one or more control device(s), which may be referred to as input control devices, for controlling the medical instrument system 614 or the imaging system 615. The control device(s) may include one or more of any number of a variety of input devices, such as hand grips, joysticks, trackballs, data gloves, trigger-guns, foot pedals, hand-operated controllers, voice recognition devices, touch screens, body motion or presence sensors, and other types of input devices.

In some embodiments, the control device(s) will be provided with the same Cartesian degrees of freedom as the medical instrument(s) of the medical instrument system 614 to provide the surgeon S with telepresence, which is the perception that the control device(s) are integral with the instruments so that the surgeon has a strong sense of directly controlling instruments as if present at the surgical site. In other embodiments, the control device(s) may have more or fewer degrees of freedom than the associated medical instruments and still provide the surgeon S with telepresence. In some embodiments, the control device(s) are manual input devices that move with six degrees of freedom, and which may also include an actuatable handle for actuating instruments (for example, for closing grasping jaw end effectors, applying an electrical potential to an electrode, delivering a medicinal treatment, and actuating other types of instruments). Therefore, the degrees of freedom and actuation capabilities of the control device(s) are mapped to the degrees of freedom and range of motion available to the medical instrument(s).

The assembly 612 supports and manipulates the medical instrument system 614 while the surgeon S views the surgical site through the operator input system 616. An image of the surgical site may be obtained by the endoscopic imaging system 615, which may be manipulated by the assembly 612. The assembly 612 may comprise multiple endoscopic imaging systems 615 and may similarly comprise multiple medical instrument systems 614 as well. The number of medical instrument systems 614 used at one time will generally depend on the diagnostic or surgical procedure to be performed and on space constraints within the operating room, among other factors. The assembly 612 may include a kinematic structure of one or more non-servo controlled links (e.g., one or more links that may be manually positioned and locked in place, generally referred to as a manipulator support structure) and a manipulator. When the manipulator takes the form of a teleoperational manipulator, the assembly 612 is a teleoperational assembly. The assembly 612 includes a plurality of motors that drive inputs on the medical instrument system 614. In an embodiment, these motors move in response to commands from a control system (e.g., control system 620). The motors include drive systems which when coupled to the medical instrument system 614 may advance a medical instrument into a naturally or surgically created anatomical orifice. Other motorized drive systems may move the distal end of said medical instrument in multiple degrees of freedom, which may include three degrees of linear motion (e.g., linear motion along the X, Y, Z Cartesian axes) and three degrees of rotational motion (e.g., rotation about the X, Y, Z Cartesian axes). Additionally, the motors may be used to actuate an articulable end effector of the medical instrument for grasping tissue in the jaws of a biopsy device or the like. Medical instruments of the medical instrument system 614 may include end effectors having a single working member such as a scalpel, a blunt blade, an optical fiber, or an electrode. Other end effectors may include, for example, forceps, graspers, scissors, or clip appliers.

The medical system 10 also includes a control system 620. The control system 620 includes at least one memory 624 and at least one processor 622 (which may be part of a processing unit) for effecting control between the medical instrument system 614, the operator input system 616, and other auxiliary systems 626 which may include, for example, imaging systems, audio systems, fluid delivery systems, display systems, illumination systems, steering control systems, irrigation systems, and/or suction systems. A clinician may circulate within the medical environment 611 and may access, for example, the assembly 612 during a set up procedure or view a display of the auxiliary system 626 from the patient bedside. In some embodiments, the auxiliary system 626 may include a display screen that is separate from the operator input system 616. In some examples, the display screen may be a standalone screen that is capable of being moved around the medical environment 611. The display screen may be orientated such that the surgeon S and one or more other clinicians or assistants may simultaneously view the display screen.

Though depicted as being external to the assembly 612 in FIG. 6, the control system 620 may, in some embodiments, be contained wholly within the assembly 612. The control system 620 also includes programmed instructions (e.g., stored on a non-transitory, computer-readable medium) to implement some or all of the methods described in accordance with aspects disclosed herein. While the control system 620 is shown as a single block in the simplified schematic of FIG. 6, the control system 620 may include two or more data processing circuits with one portion of the processing optionally being performed on or adjacent the assembly 612, another portion of the processing being performed at the operator input system 616, and the like.

Any of a wide variety of centralized or distributed data processing architectures may be employed. Similarly, the programmed instructions may be implemented as a number of separate programs or subroutines, or they may be integrated into a number of other aspects of the systems described herein, including teleoperational systems. In one embodiment, the control system 620 supports wireless communication protocols such as Bluetooth, IrDA, HomeRF, IEEE 802.11, DECT, and Wireless Telemetry.

The control system 620 is in communication with a database 627 which may store one or more medical procedure records. The database 627 may be stored in the memory 624 and may be dynamically updated. Additionally or alternatively, the database 627 may be stored on a device such as a server or a portable storage device that is accessible by the control system 620 via an internal network (e.g., a secured network of a medical facility or a teleoperational system provider) or an external network (e.g., the Internet). The database 627 may be distributed throughout two or more locations. For example, the database 627 may be present on multiple devices which may include the devices of different entities and/or a cloud server. Additionally or alternatively, the database 627 may be stored on a portable user-assigned device such as a computer, a mobile device, a smart phone, a laptop, an electronic badge, a tablet, a pager, and other similar user devices.

In some embodiments, the control system 620 may include one or more servo controllers that receive force and/or torque feedback from the medical instrument system 614. Responsive to the feedback, the servo controllers transmit signals to the operator input system 616. The servo controller(s) may also transmit signals instructing assembly 612 to move the medical instrument system(s) 14 and/or endoscopic imaging system 615 which extend into an internal surgical site within the patient body via openings in the body. Any suitable conventional or specialized servo controller may be used. A servo controller may be separate from, or integrated with, assembly 612. In some embodiments, the servo controller and assembly 612 are provided as part of a teleoperational arm cart positioned adjacent to the patient's body.

The control system 620 can be coupled with the endoscopic imaging system 615 and can include a processor to process captured images for subsequent display, such as to a surgeon on the surgeon's control console, or on another suitable display located locally and/or remotely. For example, where a stereoscopic endoscope is used, the control system 620 can process the captured images to present the surgeon with coordinated stereo images of the surgical site as a field of view image. Such coordination can include alignment between the opposing images and can include adjusting the stereo working distance of the stereoscopic endoscope.

In alternative embodiments, the medical system 610 may include more than one assembly 612 and/or more than one operator input system 616. The exact number of assemblies 612 will depend on the surgical procedure and the space constraints within the operating room, among other factors. The operator input systems 616 may be collocated or they may be positioned in separate locations. Multiple operator input systems 616 allow more than one operator to control one or more assemblies 612 in various combinations. The medical system 610 may also be used to train and rehearse medical procedures.

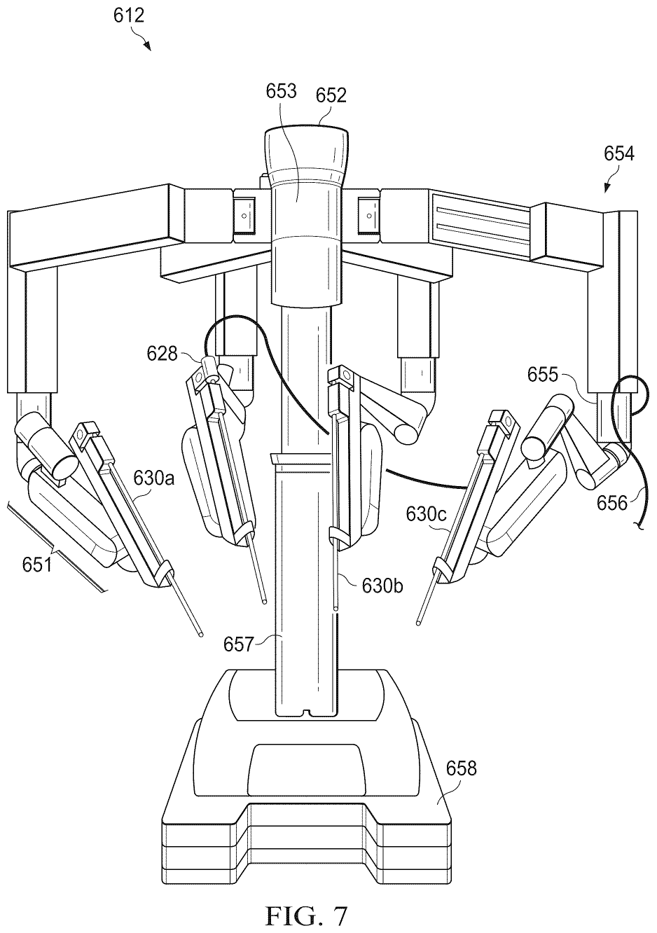

FIG. 7 is a perspective view of one embodiment of an assembly 612 which may be referred to as a manipulating system, patient side cart, surgical cart, teleoperational arm cart, or surgical robot. The assembly 612 shown provides for the manipulation of three surgical tools 630a, 630b, and 630c (e.g., medical instrument systems 614) and an imaging device 628 (e.g., endoscopic imaging system 615), such as a stereoscopic endoscope used for the capture of images of the site of the procedure. The imaging device 628 may transmit signals over a cable 656 to the control system 620. Manipulation is provided by teleoperative mechanisms having a number of joints. The imaging device 628 and the surgical tools 630a-c can be positioned and manipulated through incisions in the patient so that a kinematic remote center is maintained at the incision to minimize the size of the incision. Images of the surgical site can include images of the distal ends of the surgical tools 630a-c when they are positioned within the field-of-view of the imaging device 628. The imaging device 628 and the surgical tools 630a-c may each be therapeutic, diagnostic, or imaging instruments.

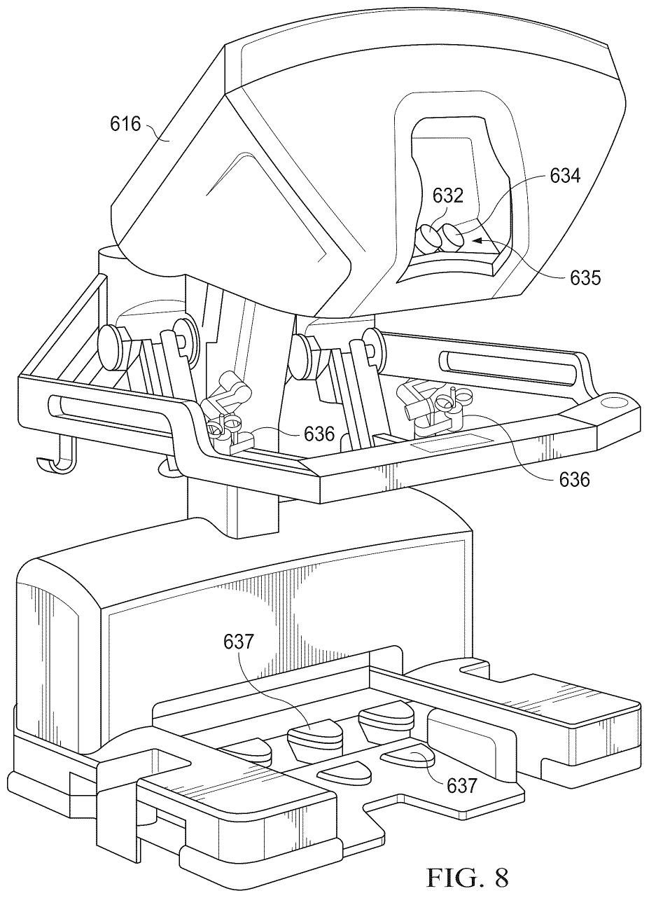

FIG. 8 is a perspective view of an embodiment of the operator input system 616 at the surgeon's control console. The operator input system 16 includes a left eye display 632 and a right eye display 634 for presenting the surgeon S with a coordinated stereo view of the surgical environment that enables depth perception. The left and right eye displays 632, 634 may be components of a display system 635. In other embodiments, the display system 35 may include one or more other types of displays. In some embodiments, image(s) displayed on the display system 635 may be separately or concurrently displayed on at least one display screen of the auxiliary system 626.

The operator input system 616 further includes one or more input control devices 636, which in turn cause the assembly 612 to manipulate one or more instruments of the endoscopic imaging system 615 and/or the medical instrument system 614. The input control devices 636 can provide the same Cartesian degrees of freedom as their associated instruments to provide the surgeon S with telepresence, or the perception that the input control devices 636 are integral with said instruments so that the surgeon has a strong sense of directly controlling the instruments. Therefore, the degrees of freedom of each input control device 636 are mapped to the degrees of freedom of each input control device's 636 associated instruments (e.g., one or more of the instruments of the endoscopic imaging system 615 and/or the medical instrument system 614.). To this end, position, force, and tactile feedback sensors (not shown) may be employed to transmit position, force, and tactile sensations from the medical instruments, e.g., the surgical tools 630a-c or the imaging device 628, back to the surgeon's hands through the input control devices 636. Additionally, the arrangement of the medical instruments may be mapped to the arrangement of the surgeon's hands and the view from the surgeon's eyes so that the surgeon has a strong sense of directly controlling the instruments. Input control devices 637 are foot pedals that receive input from a user's foot. Aspects of the operator input system 616, the assembly 612, and the auxiliary systems 626 may be adjustable and customizable to meet the physical needs, skill level, or preferences of the surgeon S.

In the description, specific details have been set forth describing some embodiments. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure.

Elements described in detail with reference to one embodiment, implementation, or application optionally may be included, whenever practical, in other embodiments, implementations, or applications in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions. Not all the illustrated processes may be performed in all embodiments of the disclosed methods. Additionally, one or more processes that are not expressly illustrated in may be included before, after, in between, or as part of the illustrated processes. In some embodiments, one or more of the processes may be performed by a control system or may be implemented, at least in part, in the form of executable code stored on non-transitory, tangible, machine-readable media that when run by one or more processors may cause the one or more processors to perform one or more of the processes.

Any alterations and further modifications to the described devices, instruments, methods, and any further application of the principles of the present disclosure are fully contemplated as would normally occur to one skilled in the art to which the disclosure relates. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative embodiment can be used or omitted as applicable from other illustrative embodiments. For the sake of brevity, the numerous iterations of these combinations will not be described separately. For simplicity, in some instances the same reference numbers are used throughout the drawings to refer to the same or like parts.

The systems and methods described herein may be suited for imaging, any of a variety of anatomic systems, including the lung, colon, the intestines, the stomach, the liver, the kidneys and kidney calices, the brain, the heart, the circulatory system including vasculature, and/or the like. While some embodiments are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

One or more elements in embodiments of this disclosure may be implemented in software to execute on a processor of a computer system such as control processing system. When implemented in software, the elements of the embodiments of this disclosure may be code segments to perform various tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and/or magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In some examples, the control system may support wireless communication protocols such as Bluetooth, Infrared Data Association (IrDA), HomeRF, IEEE 802.11, Digital Enhanced Cordless Telecommunications (DECT), ultra-wideband (UWB), ZigBee, and Wireless Telemetry.

Note that the processes and displays presented might not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the embodiments of the invention are not described with reference to any particular programming language. It will be appreciated that a variety of programming languages may be used to implement the teachings of the invention as described herein.

This disclosure describes various instruments, portions of instruments, and anatomic structures in terms of their state in three-dimensional space. As used herein, the term position refers to the location of an object or a portion of an object in a three-dimensional space (e.g., three degrees of translational freedom along Cartesian x-, y-, and z-coordinates). As used herein, the term orientation refers to the rotational placement of an object or a portion of an object (e.g., in one or more degrees of rotational freedom such as roll, pitch, and/or yaw). As used herein, the term pose refers to the position of an object or a portion of an object in at least one degree of translational freedom and to the orientation of that object or portion of the object in at least one degree of rotational freedom (e.g., up to six total degrees of freedom). As used herein, the term shape refers to a set of poses, positions, or orientations measured along an object.

While certain illustrative embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art.

1-31. (canceled)

32. A robot-assisted medical system comprising:
a manipulator arm;
an operator console;
a medical instrument coupled to the manipulator arm; and
a control system in communication with the operator console and the manipulator arm, wherein the control system comprises:
a processor; and
a memory comprising machine readable instructions that, when executed by the processor, cause the control system to:
receive a plurality of medical procedure records;
identify a set of characteristic actions common among the plurality of medical procedure records; and
determine at least one parameter for each characteristic action in the set of characteristic actions.

33. (canceled)

34. The robot-assisted medical system of claim 32, wherein each of the plurality of medical procedure records includes medical procedure information for a medical procedure performed on a unique patient.

35. The robot-assisted medical system of claim 32, wherein each of the plurality of medical procedure records includes medical procedure information for a medical procedure performed by a common medical practitioner.

36. The robot-assisted medical system of claim 32, wherein each of the plurality of medical procedure records includes medical procedure information for a medical procedure performed by a guide medical practitioner.

37. The robot-assisted medical system of claim 32, wherein the at least one parameter includes a measure of an interventional consequence.

38. The robot-assisted medical system of claim 32, wherein the set of characteristic actions includes an action of the medical instrument coupled to the manipulator arm, the medical instrument common among the plurality of medical procedure records.

39. The robot-assisted medical system of claim 38, wherein the action of the medical instrument is a rotational motion of a wrist joint of the medical instrument.

40. The robot-assisted medical system of claim 39, wherein the at least one parameter includes a measure of amount rotation of the wrist joint.

41. The robot-assisted medical system of claim 39, wherein the at least one parameter includes a duration of time to complete the rotational motion.

42. The robot-assisted medical system of claim 38, wherein the action of the medical instrument is a clamping action of an end effector of the medical instrument.

43. The robot-assisted medical system of claim 42, wherein the at least one parameter includes a measure of force applied by the clamping action of the end effector.

44. The robot-assisted medical system of claim 42, wherein the at least one parameter includes a duration of time to complete the clamping action.

45. The robot-assisted medical system of claim 38, wherein the action of the medical instrument is delivery of energy by the medical instrument.

46. The robot-assisted medical system of claim 45, wherein the at least one parameter includes a measure of energy applied by medical instrument.

47. The robot-assisted medical system of claim 45, wherein the at least one parameter includes a duration of time for the delivery of energy by the medical instrument.

48. The robot-assisted medical system of claim 38, wherein the medical instrument is an imaging instrument, and the action of the medical instrument is a pose change of the imaging instrument.

49. The robot-assisted medical system of claim 32, wherein the set of characteristic actions includes an action of the manipulator arm, wherein the manipulator arm is common among the plurality of medical procedure records.

50. The robot-assisted medical system of claim 49, wherein the at least one parameter includes a measure of an amount motion of the manipulator arm or a pose of manipulator arm during an arm collision incident.

51. The robot-assisted medical system of claim 32, wherein the set of characteristic actions includes an action at the operator console, wherein the operator console is common among the plurality of medical procedure records.

52. The robot-assisted medical system of claim 51, wherein the at least one parameter includes a duration of head disengagement from the operator console or a measure of motion of an operator input device at the operator console.
